(19) United States
(12) Patent Application Publication
(10) Pub. No.: US 2013/0273183 A1
(43) Pub. Date: Oct. 17, 2013

(54) 2-Pyrimidine Thioesters and Thiocarbonates as Skin Brightening Agents

(71) Applicants: Stephanie Kay Clendennen, Kingsport, TN (US); Liu Deng, Kingsport, TN (US); Neil Warren Boaz, Kingsport, TN (US)

(72) Inventors: Stephanie Kay Clendennen, Kingsport, TN (US); Liu Deng, Kingsport, TN (US); Neil Warren Boaz, Kingsport, TN (US)

(73) Assignee: EASTMAN CHEMICAL COMPANY, Kingsport, TN (US)

(21) Appl. No.: 13/446,240

(22) Filed: Apr. 13, 2012

(51) Int. Cl. A61K 8/49; A61K 8/58; A61Q 19/02; A61K 8/97; A61K 8/92; A61K 8/67

(52) U.S. Cl. 424/744; 514/274; 514/161; 514/263.34; 514/188

(57) ABSTRACT

2-Pyrimidine thioesters and thiocarbonates are disclosed as effective skin brightening agents. These compounds may be formulated with dermatologically acceptable carriers to form skin brightening compositions. Methods for brightening skin and for inhibiting melanogenesis using these agents are also disclosed.

### FIELD OF THE INVENTION

This invention generally relates to the field of cosmetic or dermatological compositions, and in particular, to compositions for lightening the color of mammalian skin.

### BACKGROUND OF THE INVENTION

Skin hyperpigmentation has been directly related to the formation of melanin, a dark pigment formed via tyrosine. Effective agents for skin brightening involve inhibition of melanin generation. There are many steps involved in melanin generation; thus, this inhibition can take many forms.

A typical method for stopping melanin formation is to inhibit the enzyme tyrosinase, which catalyzes the initial steps in tyrosine to melanin conversion. Effective tyrosinase inhibitors may inhibit melanin formation and are used to reduce undesirable skin pigmentation (e.g., skin brightening and/or evening out skin tone and/or reducing the appearance of age spots).

Currently, several skin brightening agents are used in the marketplace. They include hydroquinone, kojic acid, and arbutin. However, many of these materials exhibit shortcomings. Often, they are not particularly effective in vivo, or they are effective, but are toxic to skin cells.

Accordingly, there is a need in the art for new inhibitors of melanin formation that are both safe and effective. The present invention addresses this need as well as others that will become apparent from the following description and the appended claims.

### SUMMARY OF THE INVENTION

The invention is as set forth in the appended claims.

Briefly, in one aspect, the invention provides a composition for brightening skin. The composition comprises:

(a) a 2-pyrimidine thioester or thiocarbonate compound having the general formula 1:

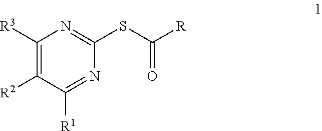

wherein

R is a substituted or unsubstituted, branched- or straight-chain, saturated, unsaturated, or polyunsaturated C₁-C₂₂ alkyl or alkoxy group; a substituted or unsubstituted C₃-C₈ cycloalkyl or cycloalkoxy group; a substituted or unsubstituted C₆-C₂₀ carbocyclic aryl or aryloxy group; or a substituted or unsubstituted C₄-C₂₀ heterocyclic group containing one or more heteroatoms selected from sulfur, nitrogen, oxygen, and mixtures thereof; and

R¹, R², and R³ are each independently selected from hydrogen; a substituted or unsubstituted, branched- or straight-chain, saturated, unsaturated, or polyunsaturated C₁-C₂₂ alkyl group, wherein the branching and/or substitution of R¹ and R² may connect to form a ring; a substituted or unsubstituted C₃-C₈ cycloalkyl group; a substituted or unsubstituted C₆-C₁₀ carbocyclic aryl group; a substituted or unsubstituted C₄-C₁₀ heterocyclic group containing one or more heteroatoms selected from sulfur, nitrogen, oxygen, and mixtures thereof; a hydroxyl group; a C₁-C₆ alkoxy group; a carboxyl group; an amino group; a C₁-C₁₅ aminocarbonyl group; a C₁-C₁₅ amido group; a cyano group; a C₂-C₆ alkoxycarbonyl group; a C₂-C₆-alkanoyloxy group; a thiol group; a thioether group; a C₂-C₁₀ dialkylamino group; a C₃-C₁₅ trialkylammonium group; and a halogen, and

(b) a dermatologically acceptable carrier.

In another aspect, the invention provides a method for brightening skin. The method comprises topically applying the skin brightening composition according to the invention to skin in need of brightening.

In yet another aspect, the invention provides a method for inhibiting melanogenesis. The method comprises contacting melanocytes with a 2-pyrimidine thioester or thiocarbonate compound having the general formula 1 defined above.

### DETAILED DESCRIPTION OF THE INVENTION

It has been surprisingly found that 2-pyrimidine thioesters and thiocarbonates can be both safe and effective as skin brightening agents.

The 2-pyrimidine thioesters and thiocarbonates according to the invention can be represented by the general formula 1:

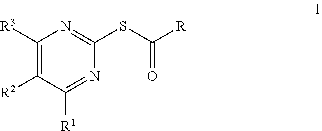

wherein

R is a substituted or unsubstituted, branched- or straight-chain, saturated, unsaturated, or polyunsaturated C₁-C₂₂ alkyl or alkoxy group; a substituted or unsubstituted C₃-C₈ cycloalkyl or cycloalkoxy group; a substituted or unsubstituted C₆-C₂₀ carbocyclic aryl or aryloxy group; or a substituted or unsubstituted C₄-C₂₀ heterocyclic group containing one or more heteroatoms selected from sulfur, nitrogen, oxygen, and mixtures thereof; and

R¹, R², and R³ are each independently selected from hydrogen; a substituted or unsubstituted, branched- or straight-chain, saturated, unsaturated, or polyunsaturated C₁-C₂₂ alkyl group, wherein the branching and/or substitution of R¹ and R² may connect to form a ring; a substituted or unsubstituted C₃-C₈ cycloalkyl group; a substituted or unsubstituted C₆-C₁₀ carbocyclic aryl group; a substituted or unsubstituted C₄-C₁₀ heterocyclic group containing one or more heteroatoms selected from sulfur, nitrogen, oxygen, and mixtures thereof; a hydroxyl group; a C₁-C₆ alkoxy group; a carboxyl group; an amino group; a C₁-C₁₅ aminocarbonyl group; a C₁-C₁₅ amido group; a cyano group; a C₂-C₆ alkoxycarbonyl group; a C₂-C₆-alkanoyloxy group; a thiol group; a thioether group; a C₂-C₁₀ dialkylamino group; a C₃-C₁₅ trialkylammonium group; and a halogen.

The terms “C₁-C₆ alkoxy,” “C₂-C₆ alkoxycarbonyl,” and “C₂-C₆ alkanoyloxy” are used to denote radicals corresponding to the structures —OR⁴, —CO₂R⁴, and —OCOR⁴, respectively, wherein R⁴ is a C₁-C₆ alkyl or a substituted C₁-C₆ alkyl.

The terms “C₁-C₁₅ aminocarbonyl” and “C₁-C₁₅ amido” are used to denote radicals corresponding to the structures —NHCOR⁵ and —CONHR⁵, respectively, wherein R⁵ is a C₁-C₁₅ alkyl or a substituted C₁-C₁₅ alkyl.

In some embodiments, the 2-pyrimidine thioesters and thiocarbonates according to the invention include those denoted by the general formula 1 wherein (i) R is selected from a substituted or unsubstituted, branched- or straight-chain saturated C₁-C₂₂ alkyl or alkoxy group; a substituted or unsubstituted, branched- or straight-chain C₂-C₂₂ alkenyl or alkenyloxy group; a substituted or unsubstituted, branched- or straight-chain C₄-C₂₂ dienyl group; a substituted or unsubstituted C₃-C₈ cycloalkyl or cycloalkoxy group; a substituted or unsubstituted C₆-C₂₀ carbocyclic aryl or aryloxy group; and a substituted or unsubstituted C₄-C₂₀ heteroaryl group, and (ii) R¹, R², and R³ are each independently selected from hydrogen and a straight- or branched-chain C₁-C₆ alkyl or alkenyl group.

In other embodiments, the 2-pyrimidine thioesters and thiocarbonates according to the invention include those denoted by the general formula 1 wherein (i) R is a C₁-C₁₀ alkyl group or alkoxy group, a C₂-C₁₀ alkenyl or alkenyloxy group, a C₄-C₁₀ dienyl group, a C₃-C₈ cycloalkyl or cycloalkoxy group, a C₆-C₁₂ carbocyclic aryl or aryloxy group, or a C₄-C₁₀ heteroaryl group; and (ii) R¹, R², and R³ are each independently selected from hydrogen and a C₁-C₆ alkyl or alkenyl group.

In yet other embodiments, the 2-pyrimidine thioesters and thiocarbonates according to the invention include those denoted by the general formula 1 wherein (i) R is as defined in any embodiment set forth above and (ii) R¹, R², and R³ are each hydrogen.

The saturated, unsaturated, and polyunsaturated alkyl, alkoxy, cycloalkyl, and cycloalkoxy groups, which R, R¹, R², and R³ may represent, may be straight- or branched-chain radicals containing up to about 22 carbon atoms and may be substituted, for example, with one to five groups selected from hydroxyl, C₁-C₆ alkoxy, carboxyl, amino, C₁-C₁₅ aminocarbonyl, C₁-C₁₅ amido, cyano, C₂-C₆ alkoxycarbonyl, C₂-C₆ alkanoyloxy, aryl, heteroaryl, thiol, thioether, C₂-C₁₀ dialkylamino, C₃-C₁₅ trialkylammonium, and halogen.

The aryl and aryloxy groups, which R, R¹, R², and R³ may represent (or any aryl substituents), include phenyl, phenyloxy, naphthyl, naphthyloxy, anthracenyl, and anthracenyloxy. The phenyl, phenyloxy, naphthyl, naphthyloxy, anthracenyl, and antracenyloxy may be substituted with one to five substituents selected from C₁-C₆ alkyl, substituted C₁-C₆ alkyl, C₆-C₁₀ aryl, substituted C₆-C₁₀ aryl, C₁-C₆ alkoxy, halogen, carboxy, cyano, C₁-C₆ alkanoyloxy, C₁-C₆ alkylthio, C₁-C₆ alkylsulfonyl, trifluoromethyl, hydroxyl, C₂-C₆ alkoxycarbonyl, C₂-C₆ alkanoylamino, —OR⁶, —S—R⁶, —SO₂—R⁶, —NHSO₂R⁶, and —NHCO₂R⁶ wherein R⁶ is phenyl or naphthyl, optionally substituted with one to three groups selected from C₁-C₆ alkyl, C₆-C₁₀ aryl, C₁-C₆ alkoxy, and halogen.

The heterocyclic groups, which R, R¹, R², and R³ may represent (or any heteroaryl substituents), include 5- or 6-membered rings containing one to three heteroatoms selected from oxygen, sulfur, and nitrogen. Examples of such heterocyclic groups include pyranyl, oxopyranyl, dihydropyranyl, oxodihydropyranyl, tetrahydropyranyl, thienyl, furyl, pyrrolyl, imidazolyl, pyrazolyl, thiazolyl, isothiazolyl, oxazolyl, isoxazolyl, triazolyl, thiadiazolyl, oxadiazolyl, tetrazolyl, pyridyl, pyrimidyl, benzoxazolyl, benzothiazolyl, benzimidazolyl, and indolyl. The heterocyclic radicals may be substituted, for example, with up to three groups such as C₁-C₆ alkyl, C₁-C₆ alkoxy, substituted C₁-C₆ alkyl, halogen, C₁-C₆ alkylthio, aryl, arylthio, aryloxy, C₂-C₆ alkoxycarbonyl, and C₂-C₆-alkanoylamino. The heterocyclic radicals may also be substituted with a fused ring system, e.g., a benzo or naphtho residue, which may be unsubstituted or substituted, for example, with up to three of the groups set forth in the preceding sentence.

The terms “halogen” and “halide” are used to include fluorine, chlorine, bromine, and iodine.

Examples of the preferred compounds of the invention include those represented by the general formula 1 wherein (i) R is methyl, ethyl, 1,1-dimethylethyl, n-pentyl, phenyl, 3-pyridyl, 4-pyridyl, or ethoxy; and (ii) R¹, R², and R³ are hydrogen.

The compounds of the general formula 1 may be prepared by various methods known in the art, such as those described in U.S. Pat. No. 3,904,612; the entire content of which is hereby incorporated by reference.

For example, the compounds of the general formula 1 may be prepared by reacting an optionally substituted 2-mercaptopyrimidine with a carboxylic acid or a reactive derivative thereof in an inert solvent. The 2-mercaptopyrimidine is a common compound and may be obtained by methods known in the art. The reactive derivative includes acyl halides, acid anhydrides, unsaturated carboxylic acids, esters of carboxylic acids and unsaturated carboxylic acids, halogenated esters, and lactones. The carboxylic acid or reactive derivative thereof may contain various substituents or properly protected functional groups identified in connection with R in the general formula 1. Examples of suitable carboxylic acids and reactive derivatives thereof include acetic anhydride, propionic anhydride, ethyl chloroformate, hexanoyl chloride, pivaloyl chloride, benzoyl chloride, nicotinoyl chloride hydrochloride, and isonicotinoyl chloride hydrochloride.

Any solvent may be used in the above reaction so long as it is substantially inert to the reactants and the reaction product, and can dissolve or suspend the reactants and/or reaction product. Examples of such solvents include dioxane, ethyl acetate, methylene chloride, chloroform, acetonitrile, ethyl ether, dichloromethane, and tetrahydrofuran.

When the reactive derivative includes a halogen such as in the case of acyl halides and halogenated esters, the reaction may be advantageously carried out in the presence of a base such as sodium or potassium hydroxide or triethylamine to avoid production and/or accumulation of a hydrogen halide by-product.

The temperature for this reaction is not critical. The reaction may be conducted in the range of −50° C. to 100° C., or at the reflux temperature of the inert solvent.

The reaction time can vary, depending on the specific reactants and the reaction temperature employed. In most cases, the reaction time can range from 30 minutes to 24 hours. The reaction time, however, is not critical and may be conducted for less than 30 minutes or over 24 hours.

After the reaction is completed, the product of the general formula 1 may be isolated using methods known to those of skill in the art, e.g., by extraction, filtration, and/or crystallization.

The 2-pyrimidine thioesters and thiocarbonates according to the invention can be advantageously employed in compositions for brightening skin as one of the active ingredients. Such compositions are typically formulated with one or more dermatologically acceptable carriers. By “dermatologically acceptable,” it is meant the carrier should cause minimal, if any, adverse effects on the skin area to which it is applied.

The carrier may be a material or combination of materials that is normally used to carry or deliver an active skin treating agent to the skin. The specific carrier material will depend upon the delivery form selected. For example, the skin brightening composition may be in the form of a lotion, cream, ointment, soap, stick, gel, foam, emulsion, dispersion, spray, etc. Each composition would typically include any of the known topical carriers or excipients necessary for obtaining the particular form.

Suitable carriers/excipients include, e.g., mineral oils and emulsifying agents. The carriers may also include water, alcohol, or water/alcohol combinations, or other solvent(s) or solvent systems in which the active agents may be solubilized, dispersed, or emulsified. Preferably, the composition would include excipients that create a substantially stable skin brightening composition and/or provide body and viscosity to the composition so that the active agents do not merely run off or evaporate from the skin once applied. Preferably, the carrier would facilitate topical application and, in some cases, provide additional benefits such as moisturizing the affected skin areas. Also preferably, the carrier would assist the composition (a) to form a film or layer on the skin to which it is applied so as to localize the application, (b) to provide some resistance to removal by contact with water and/or perspiration, and/or (c) to aid in the percutaneous delivery of the active agents.

Preferably, the carrier has a minimal, if any, deactivating or oxidizing effect on the active ingredients.

As noted, examples of suitable carriers/excipients include oils, alcohols, and emollients. Specific examples of such ingredients include olive oil; hydrocarbon oils and waxes; silicone oils; other vegetable, animal, or marine fats or oils; glyceride derivatives; fatty acids; fatty acid esters or alcohols or alcohol ethers; lecithin; lanolin and derivatives; polyhydric alcohols or esters; wax esters; sterols; and phospholipids.

Additional examples of carriers/excipients include emulsifiers and surfactants. Suitable surfactants can include anionic surfactants (such as alcohol ether sulfates, linear alkylbenzene sulfonates, and acyl isethionates), cationic surfactants (such as quaternary ammonium salts, fatty amine oxides, and ester quats), and non-ionic surfactants (such as alky polyglycosides, alcohol ethoxylates, and fatty alcanol amides).

Other components that may be included in the skin brightening compositions of the invention include conditioning agents (such as polyquaterniums and panthenol), pearlizing agents (such as glycol distearate, distearyl ether, and mica), UV filters (such as octocrylene, octyl methoxycinnamate, benzophenone-4, titanium dioxide, and zinc oxide), exfoliation additives (such as apricot seeds, walnut shells, polymer beads, and pumice), silicones (such as dimethicone cyclomethicone, and amodimethicone), moisturizing agents (such as petrolatum, sunflower oil, fatty alcohols, and shea butter), foam stabilizers (such as cocamide MEA and cocamide DEA), anti-bacterial agents (such as triclosan), humectants (such as glycerin), thickening agents (such as guar, sodium chloride, and carbomer), hair and skin damage repair agents (such as proteins, hydrolyzed proteins, and hydrolyzed collagen), and foam boosters (such as cocamide MIPA).

The skin brightening compositions of the invention may also contain other skin care or cosmetic ingredients such as retinol, retinyl esters, tetronic acid, tetronic acid derivatives, hydroquinone, kojic acid, gallic acid, arbutin, 4-hydroxybenzyl alcohol and esters, α-hydroxy acids, niacinamide, pyridoxine, ascorbic acid, vitamin E and derivatives, aloe, salicylic acid, benzoyl peroxide, witch hazel, caffeine, zinc pyrithione, and fatty acid esters of ascorbic acid. The compositions of the invention may also include various other ingredients typically associated with skin care or cosmetic products. Such other ingredients are known to those of skill in the art.

By using different combinations and proportions of the carrier/excipient, the compositions of the invention can be formulated into various useful forms such as a lotion, cream, gel, solid stick, spray, etc.

It should be understood that the specific ingredients mentioned above are merely illustrative and that some embodiments of the compositions of the present invention may include other suitable components and agents. The compositions of the invention may be used for, among other things, cosmetic and/or skin care purposes and may be formulated with different ingredients according to the desired use. The compositions can also be incorporated into plasters, bandages, dressings, gauze pads, and similar articles.

Typical compositions of the invention can contain from 0.001% to 20% by weight, from 0.01% to 10% by weight, or from about 0.1% to about 5% by weight, of the 2-pyrimidine thioesters or thiocarbonates according to the general formula 1. Lower concentrations may be employed for less pronounced conditions (e.g., hyperpigmentation and in sunscreens and sunblocks used after skin brightening treatment) and higher concentrations may be employed for more acute conditions.

The skin brightening compositions of the present invention may be prepared by any method known in the art for cosmetic and/or dermatological preparations. Generally, the method comprises mixing the components. On occasion, especially where insoluble or immiscible components are employed, higher agitation or homogenization may be necessary to prepare an appropriate composition, e.g., an emulsion or suspension, etc. Additionally, during the preparation, it may be desirable to add pH adjusters and/or buffers in order to maintain a proper pH of the composition for topical application, especially if very acidic or basic ingredients are employed. Generally, the pH should be close to neutral or slightly on the acidic side, possibly as low as pH 4. More desirably, the pH will be in the range of 5 to 6.5.

In a second aspect, the invention relates to a method for brightening skin. The method includes the step of topically applying the compositions of the invention to skin in need of brightening, for example, such as darker skin areas on a subject. The darker skin areas can be in the form of spots, blotches, or relatively large areas of darker color. The “skin in need of brightening” may not necessarily be an actual need, but may be a perceived need, for example, by an individual with a subjective belief that his/her skin shade is darker than desired. All of the descriptions of the 2-pyrimidine thioesters and thiocarbonates, the dermatologically acceptable carrier, other ingredients, and concentration of the 2-pyrimidine thioesters and thiocarbonates in the compositions of the invention described above, apply to this aspect of the invention as well.

The skin brightening compositions of the invention may be applied to the skin, for example, by spraying or rubbing, in a predetermined regimen (e.g., one to four times per day for a month or more) or on an as-needed basis. Generally, gradual brightening can be expected with each successive application. Insofar as has been determined based upon in vitro studies, no adverse side effects are encountered.

In a third aspect, the invention provides a method for inhibiting melanogenesis. The method includes the step of contacting melanocytes with a 2-pyrimidine thioester or thiocarbonate compound having the general formula 1. The amount of the 2-pyrimidine thioester or thiocarbonate compound effective for inhibiting melanogenesis may vary over a wide range. For example, it has been found that two 3-mL doses of 0.1 mmol/L and 1.0 mmol/L solutions of a 2-pyrimidine thioester or thiocarbonate compound dissolved in dimethylsulfoxide in a 24-hour period were effective to treat a population of 3×10⁵ melanocyte cells.

As used herein, the indefinite articles “a” and “an” mean one or more, unless the context clearly suggests otherwise. Similarly, the singular form of nouns includes their plural form, and vice versa, unless the context clearly suggests otherwise.

This invention can be further illustrated by the following working examples, although it should be understood that these examples are included merely for purposes of illustration and are not intended to limit the scope of the invention. Unless otherwise indicated or the context suggests differently, all percentages are by weight.

### EXAMPLES

The structures of the compounds prepared in Examples 1-8 and Comparative Examples 1 and 2 are shown in Table 1 below.

TABLE 1

| | |
|---|---|
| Example 1 | 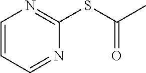 |
| Example 2 | 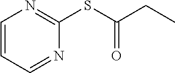 |
| Example 3 | 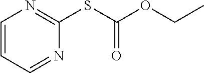 |
| Example 4 | 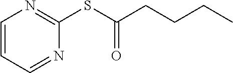 |
| Example 5 | 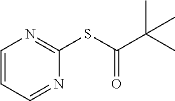 |
| Example 6 | 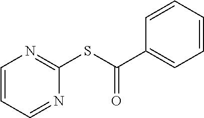 [SMILES: O=C(Sc1ncccn1)c1ccccc1] |
| Example 7 | 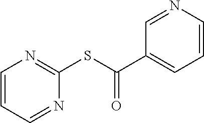 [SMILES: O=C(Sc1ncccn1)c1cccnc1] |
| Example 8 | 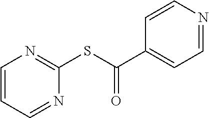 [SMILES: O=C(Sc1ncccn1)c1ccncc1] |
| Comparative Example 1 | 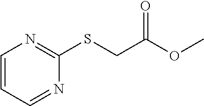 |
| Comparative Example 2 | 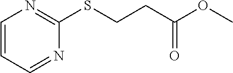 |

### Example 1

### Preparation of Pyrimidin-2-yl Thiolacetate

2-Mercaptopyrimidine (1.0 g, 8.92 mmol, 1 eq), triethylamine (1.8 g, 17.83 mmol, 2.0 eq), and acetonitrile (25 mL) were added to a 100-mL, 3-neck round bottom flask equipped with a stir bar, N₂ bubbler, rubber septum, and glass stopper. The flask was purged well. Acetic anhydride (1.05 g, 10.29 mmol, 1.15 eq) was added using a gas tight syringe. The solution, which was now homogeneous, was stirred at room temperature. After one hour, thin layer chromatography (TLC) (2% MeOH/CH₂Cl₂) showed some 2-mercaptopyrimidine on the baseline. The mixture was stirred an additional hour; TLC was the same. The solution was transferred to a separatory funnel using EtOAc and water (50 mL of each), and the layers were separated. The separation was very slow, and it was necessary to add 10 mL of brine. The aqueous layer was washed with 25 mL of EtOAc. The combined organic layers were dried over MgSO₄ and concentrated. The material was run through a plug of silica using 3% MeOH/CH₂Cl₂ and concentrated to afford 1.12 g of a yellow solid (81% yield). ¹H NMR (CDCl₃) δ (ppm): 8.80-8.81 (d, J=6 Hz, 2H), 7.27-7.31 (t, J=6 Hz, 1H), 2.52 (s, 3H).

### Example 2

### Preparation of Pyrimidin-2-yl Thiolpropionate

2-Mercaptopyrimidine (1.0 g, 8.92 mmol, 1 eq), triethylamine (1.8 g, 17.83 mmol, 2.0 eq), and acetonitrile (25 mL) were added to a 100-mL, 3-neck round bottom flask equipped with a stir bar, N₂ bubbler, rubber septum, and glass stopper. The flask was purged well. Propionic anhydride (1.16 g, 8.91 mmol, 1 eq) was added using a gas tight syringe. The solution, which was now homogeneous, was stirred at room temperature. The reaction was followed by TLC (2% MeOH/CH₂Cl₂), until there was no more conversion (about 1 hour). The solution was transferred to a separatory funnel using EtOAc and water (40 mL of each), and the layers were separated. The separation was very slow, and it was necessary to add 10 mL of brine. The aqueous layer was washed with 50 mL of EtOAc. The combined organic layers were dried over MgSO₄ and concentrated. The material was run through a plug of silica using 1-2% MeOH/CH₂Cl₂ and concentrated to afford 0.79 g of a yellow biphasic mixture of liquids and solids (53% yield). ¹H NMR (CD₂Cl₂) δ (ppm): 8.75-8.77 (d, J=6 Hz, 2H), 7.27-7.30 (t, J=6 Hz, 1H), 2.73-2.81 (q, J=9 Hz, 2H), 1.18-1.23 (t, J=6 Hz, 3H).

### Example 3

### Preparation of O-Ethyl-5-pyrimidin-2-yl Monothiolcarbonate

2-Mercaptopyrimidine (1.0 g, 8.92 mmol, 1 eq), triethylamine (1.8 g, 17.83 mmol, 2.0 eq), and ethyl ether (15 mL) were added to a 100-mL, 3-neck round bottom flask equipped with a stir bar, N₂ bubbler, rubber septum, and glass stopper. The flask was purged well. Ethyl chloroformate (1.16 g, 10.7 mmol, 1.2 eq) was added using a gas tight syringe. The mixture was stirred at room temperature to afford a significant amount of precipitate. The reaction was followed by TLC until all of the 2-mercaptopyrimidine was consumed. The mixture was then transferred to a separatory funnel using EtOAc and water (25 mL of each), and the layers were separated. The aqueous layer was washed with 25 mL of EtOAc. The combined organic layers were dried over MgSO₄ and concentrated to afford a crude material. The material was run through a plug of silica using 2% MeOH/CH₂Cl₂ and concentrated to afford 1.06 g of a yellow liquid (65% yield). ¹H NMR (CD₂Cl₂) δ (ppm): 8.72-8.74 (d, J=6 Hz, 2H), 7.25-7.29 (t, J=6 Hz, 1H), 4.29-4.36 (q, J=6 Hz, 2H), 1.29-1.34 (t, J=6 Hz, 3H).

### Example 4

### Preparation of Pyrimidin-2-yl Hexanethioate

2-Mercaptopyrimidine (1.0 g, 8.92 mmol, 1 eq), triethylamine (1.81 g, 17.9 mmol, 2.0 eq), and dichloromethane (25 mL) were added to a 100-mL, 3-neck round bottom flask equipped with a stir bar, N₂ bubbler, rubber septum, and glass stopper. The flask was purged well. Hexanoyl chloride (1.32 g, 9.81 mmol, 1.1 eq) was added using a gas tight syringe. The solution was stirred at room temperature overnight. TLC (2% MeOH/CH₂Cl₂) showed a very faint spot on the baseline for 2-mercaptopyrimidine. The solution was transferred to a separatory funnel using EtOAc and water (25 mL of each), and the layers were separated. The organic layer was dried over MgSO₄ and concentrated. TLC showed spots above and below the product spot. The material was run through a 40 g preformed silica column using 2% MeOH/CH₂Cl₂. Only 0.52 g of a biphasic mixture of liquid and solids were collected (28% yield). ¹H NMR (CD₂Cl₂) δ (ppm): 8.75-8.77 (d, J=6 Hz, 2H), 7.26-7.30 (t, J=6 Hz, 1H), 2.7-2.75 (t, J=6 Hz, 2H), 1.66-1.73 (m, 2H), 1.30-1.37 (m, 4H), 0.88-0.92 (m, 3H).

### Example 5

### Preparation of Pyrimidin-2′-yl 2,2-Dimethylpropanethioate

2-Mercaptopyrimidine (1.69 g, 15.07 mmol, 1 eq), triethylamine (3.05 g, 30.1 mmol, 2.0 eq), and methylene chloride (25 mL) were added to a 100-mL, 3-neck round bottom flask equipped with a stir bar, N₂ bubbler, rubber septum, and glass stopper. The flask was purged well. Pivaloyl chloride (2.0 g, 16.59 mmol, 1.1 eq) was added using a gas tight syringe. There was a slight exotherm. The mixture was stirred at room temperature overnight. The mixture was transferred to a separatory funnel using CH₂Cl₂ and water (50 mL of each), and the layers were separated. The organic layer was dried over MgSO₄ and concentrated. The material was run through a plug of silica using 2% MeOH/CH₂Cl₂. Three fractions were collected. The first fraction had only one spot, but the ¹H NMR showed about 5.3% of the pivaloyl chloride or the acid. This fraction was pumped down three times with toluene. The impurity was now only 2% by NMR. Toluene was added to the sample, and it was put on high vacuum for 3 hours to afford 1.5 g of white solid with no impurity (yield 51%). ¹H NMR (CD₂Cl₂) δ (ppm): 8.76-8.77 (d, J=6 Hz, 2H), 7.26-7.30 (t, J=6 Hz, 1H), 1.31 (s, 9H).

### Example 6

### Preparation of Pyrimidin-2-yl Thiolbenzoate

2-Mercaptopyrimidine (1.0 g, 8.92 mmol, 1 eq), triethylamine (1.8 g, 17.83 mmol, 2.0 eq), and methylene chloride (25 mL) were added to a 100-mL, 3-neck round bottom flask equipped with a stir bar, N₂ bubbler, rubber septum, and glass stopper. The flask was purged well. Benzoyl chloride (1.38 g, 9.82 mmol, 1.1 eq) was added using a gas tight syringe. The mixture was stirred at room temperature over the weekend. The mixture was transferred to a separatory funnel using EtOAc and water (25 mL of each), and the layers were separated. The organic layer was washed with 25 mL water. The combined organic layers were dried over MgSO₄ and concentrated. TLC (2% MeOH/CH₂Cl₂) showed two spots above the product spot. The material was run through a plug of silica using CH₂Cl₂ to rid of the top two spots and then 1% MeOH/CH₂Cl₂ to collect the large spot. The latter fraction was concentrated to afford 1.53 g of a yellow oil (79% yield). ¹H NMR (CD₂Cl₂) δ (ppm): 8.81-8.83 (d, J=6 Hz, 2H), 7.97-8.0 (m, 2H), 7.63-7.68 (m, 1H), 7.49-7.55 (m, 2H), 7.33-7.36 (t, J=6 Hz, 1H).

### Example 7

### Preparation of S-Pyrimidin-2′-yl Pyridine-3-carbothioate

2-Mercaptopyrimidine (1.122 g, 10 mmol) and triethylamine (1.8 mL, 15 mmol) were mixed in anhydrous THF (50 mL). Nicotinoyl chloride hydrochloride (2.670 g, 15 mmol) was added portionwise. After stirring overnight, the reaction mixture was diluted with dichloromethane, washed with brine twice, dried, and concentrated to give the product as a pale yellow solid (1.479 g, 68%). ¹H NMR (CDCl₃) δ (ppm): 9.17 (m, 1), 8.84 (d, J=4.8 Hz, 2H), 8.83 (m, 1H), 8.25 (td, J=8.1, 2.1 Hz, 1H), 7.48 (dd, J=8.1, 0.9 Hz, 1H), 7.38 (t, J=4.8 Hz, 1H).

### Example 8

### Preparation of S-Pyrimidin-2′-yl Pyridine-4-carbothioate

2-Mercaptopyrimidine (1.122 g, 10 mmol) and triethylamine (2.1 mL, 15 mmol) were mixed in anhydrous THF (50 mL). Isonicotinoyl chloride hydrochloride (2.670 g, 15 mmol) was added portionwise. After stirring overnight, the reaction mixture was diluted with dichloromethane, washed with brine twice, dried, and concentrated to give the product as a yellow solid (1.220 g, 56%). ¹H NMR (CDCl₃) δ (ppm): 8.9-8.8 (m, 4H), 7.78 (dd, J=1.8, 4.5 Hz, 2H), 7.40 (t, J=4.8 Hz, 1H).

### Comparative Example 1

### Preparation of Methyl 2-(pyrimidin-2′-ylthio)acetate

2-Mercaptopyrimidine (1.1 g, 9.81 mmol, 1 eq), triethylamine (1.81 g, 17.9 mmol, 1.82 eq), and acetonitrile (25 mL) were added to a 100-mL, 3-neck round bottom flask equipped with a stir bar, N₂ bubbler, rubber septum, and glass stopper. The flask was purged well. Methyl 2-bromoacetate (1.67 g, 10.92 mmol, 1.1 eq) was added using a gas tight syringe. The solution was stirred at room temperature. The solution went homogeneous when the methyl bromoacetate was added, then started to lose its yellow color, and then very quickly started to form precipitate. TLC (2% MeOH/CH₂Cl₂) showed no 2-mercaptopyrimidine. The solution was transferred to a separatory funnel using EtOAc and water (25 mL of each). The layers were separated and the aqueous layer was washed with 25 mL of EtOAc. The combined organic layers were dried over Na₂SO₄ and concentrated to afford 1.64 g of an oily substance with solids in it. The material was run through a plug of silica using 2% MeOH/CH₂Cl₂ and concentrated to afford a clear yellow oil. ¹H NMR (CDCl₃) δ (ppm): 8.51-8.53 (d, J=6 Hz, 2H), 6.98-7.01 (t, J=6 Hz, 1H), 3.96 (s, 2H), 3.76 (s, 3H).

### Comparative Example 2

### Preparation of Methyl 3-(Pyrimidin-2′-ylthio)propionate

2-Mercaptopyrimidine (1.0 g, 8.92 mmol, 1 eq), triethylamine (0.09 g, 0.89 mmol, 0.1 eq), and methylene chloride (25 mL) were added to a 100-mL, 3-neck round bottom flask equipped with a stir bar, N₂ bubbler, rubber septum, and glass stopper. The flask was purged well. Methyl acrylate (0.84 g, 9.76 mmol, 1.1 eq) was added using a gas tight syringe. The mixture was stirred at room temperature for 3 days. The reaction mixture was concentrated, swirled in methylene chloride, and filtered to remove the solid 2-mercaptopyrimidine. TLC of the filtrate showed one main spot with a spot above and two faint spots below. The material was run through an 80 g preformed silica column using 1% MeOH/CH₂Cl₂ to afford 0.46 g of a slightly colored liquid (26% yield). ¹H NMR (CD₂Cl₂) δ (ppm): 8.49-8.51 (d, J=6 Hz, 2H), 6.97-7.0 (t, J=6 Hz, 1H), 3.67 (s, 3H), 3.33-3.37 (t, J=6 Hz, 2H), 2.77-2.81 (t, J=6 Hz, 2H).

### Example 9

### Cell Culture and Melanogenesis Inhibition Assay

Melanocytes (B16-F10/CRL-6475 murine melanoma cells, purchased from ATCC) were cultured in Dulbecco's Modified Eagle's Medium (DMEM)/Ham's F-12 50:50 mix with L-glutamine w/out phenol red/10% (v/v) newborn calf serum/1% (v/v) 100× Invitrogen Cat #15240-062 Antibiotic-Antimycotic at 37° C. (i.e., the growth medium) in a humidified atmosphere with 5% CO₂. The cells were seeded into 12.5 cm² tissue culture flasks at a concentration of 1.0×10⁵ cells/mL. A 3-mL cell suspension (seed medium) was added to each flask.

The test compounds were dissolved in dimethyl sulfoxide (DMSO) at a concentration of 0.5 M. This 0.5 M stock was used to prepare various concentrations of the test compounds in growth medium, with the final concentration of DMSO at 0.2% by weight. Growth medium with diluted test compound is referred to as the dose medium.

The controls were treated in the same manner, but without the test compound.

After 24 hrs of incubation, the seed medium was replaced with 3 mL of dose medium (growth medium with various concentrations of test samples or control compounds). The dose medium was replaced after 1 day, and the incubation continued for an additional 2 days. The dose medium was removed. The cells were dissolved in 1 mL of 1 N NaOH during incubation at 80° C. for 10 min. Samples of extracted melanin were transferred to a 96-well plate, and the absorbance was read at 450 nm.

Each experiment was repeated three times.

Kojic acid, arbutin, and hydroquinone were used as positive controls.

### Cell Viability Assay

MTT (3-[4,5-dimethylthiazol-2-yl]-2,5-diphenyl tetrazolium bromide) assay was conducted to measure the viability (level of metabolic activity) of living cells as a function of mitochondrial dehydrogenase activity.

After incubating the melanocytes with test samples or control compounds as mentioned above, 330 μL of a MTT solution (5 mg/mL MTT in standard medium with no added serum or antibiotic) was added. The flasks were incubated for 30 min at 37° C. in a humidified atmosphere with 5% CO₂ before the medium was carefully removed and 3.3 mL of MTT solvent (50 mL 1N HCl+450 mL isopropanol) was added.

After orbital shaking for 10 min at room temperature, 1 mL of the contents of each flask was transferred to microfuge tubes and centrifuged for 10 min. 200 μL of each tube were then transferred to a 96-well plate, and the relative extent of MTT reduction was determined based on the absorbance at 590 nm.

Each experiment was repeated three times.

Kojic acid, arbutin, and hydroquinone were used as positive controls.

Table 2 below summaries the effects of derivatives of 2-pyrimidine on melanogenesis and cell viability in B16-F10 murine melanoma cells.

TABLE 2

| | Melanogenesis | | | | Cell Viability | | | |
|---|---|---|---|---|---|---|---|---|
| | 0.1 mmol/L | | 1.0 mmol/L | | 0.1 mmol/L | | 1.0 mmol/L | |
| | % of | Standard | % of | Standard | % of | Standard | % of | Standard |
| Compounds | control | deviation | control | deviation | control | deviation | control | deviation |
| Hydroquinone | | | 33* | 8.5* | | | 3* | 0.09* |
| Kojic acid | 94 | 2.5 | 86 | 0.8 | 116 | 0.66 | 92 | 1.9 |
| Arbutin | 92 | 9.3 | 83 | 4.2 | 125 | 14 | 134 | 1.4 |
| 2-Mercapto- | 42** | 1.2** | 35* | 1.9* | 81** | NR | 68* | NR |
| Pyrimidine | | | | | | | | |
| Example 1 | 71 | 2.2 | 33 | 0.80 | 100 | 13.6 | 6.6 | 1.0 |
| Example 2 | 67 | 6.5 | 36 | 3.0 | 113 | 3.7 | 13.6 | 3.0 |
| Example 3 | 81 | 2.2 | 36 | 2.2 | 87 | 2.6 | 32 | 16 |
| Example 4 | 71 | 6.1 | 24 | 4.3 | 72 | 1.4 | 30 | 8.8 |
| Example 5 | 66 | 2.7 | 28 | 1.6 | 91 | 1.2 | 7 | 0.04 |
| Example 6 | 69 | 1.3 | 39 | 4.7 | 91 | 3.4 | 69 | 1.2 |
| Example 7 | 72 | 3.8 | 27 | 4.4 | 101 | 2.0 | 3.5 | 0.20 |
| Example 8 | 70 | 1.6 | 38* | 5.0* | 92 | 5.6 | 36* | 21* |
| Comparative | 115 | 6.8 | 98 | 3.2 | 114 | 8.8 | 117 | 4.5 |
| Example 1 | | | | | | | | |
| Comparative | 98 | 2.1 | 96* | 3.8* | 88 | 1.9 | 84* | 1.1* |
| Example 2 | | | | | | | | |

*results at 0.5 mmol/L concentration;
**results at 0.25 mmol/L concentration; and
NR: no replication.

The invention has been described in detail with particular reference to preferred embodiments thereof, but it will be understood that variations and modifications can be effected within the spirit and scope of the invention.

We claim:

1. A composition for brightening skin comprising:
(a) a 2-pyrimidine thioester or thiocarbonate compound having the general formula 1: 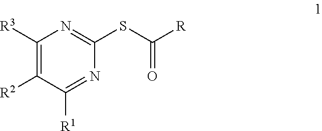
wherein
R is selected from the group consisting of a substituted or unsubstituted, branched- or straight-chain, saturated, unsaturated, or polyunsaturated C₁-C₂₂ alkyl or alkoxy group; a substituted or unsubstituted C₃-C₈ cycloalkyl or cycloalkoxy group; a substituted or unsubstituted C₆-C₂₀ carbocyclic aryl or aryloxy group; and a substituted or unsubstituted C₄-C₂₀ heterocyclic group containing one or more heteroatoms selected from the group consisting of sulfur, nitrogen, and oxygen; and
R¹, R², and R³ are each independently selected from the group consisting of hydrogen; a substituted or unsubstituted, branched- or straight-chain, saturated, unsaturated, or polyunsaturated C₁-C₂₂ alkyl group, wherein the branching and/or substitution of R¹ and R² may connect to form a ring; a substituted or unsubstituted C₃-C₈ cycloalkyl group; a substituted or unsubstituted C₆-C₁₀ carbocyclic aryl group; a substituted or unsubstituted C₄-C₁₀ heterocyclic group containing one or more heteroatoms selected from the group consisting of sulfur, nitrogen, and oxygen; a hydroxyl group; a C₁-C₆ alkoxy group; a carboxyl group; an amino group; a C₁-C₁₅ aminocarbonyl group; a C₁-C₁₅ amido group; a cyano group; a C₂-C₆ alkoxycarbonyl group; a C₂-C₆-alkanoyloxy group; a thiol group; a thioether group; a C₂-C₁₀ dialkylamino group; a C₃-C₁₅ trialkylammonium group; and a halogen, and
(b) a dermatologically acceptable carrier.

2. The composition according to claim 1, wherein
R is selected from the group consisting of a substituted or unsubstituted, branched- or straight-chain saturated C₁-C₂₂ alkyl group or alkoxy group; a substituted or unsubstituted, branched- or straight-chain C₂-C₂₂ alkenyl or alkenyloxy group; a substituted or unsubstituted, branched- or straight-chain C₄-C₂₂ dienyl group; a substituted or unsubstituted C₃-C₈ cycloalkyl or cycloalkoxy group; a substituted or unsubstituted C₆-C₂₀ carbocyclic aryl or aryloxy group; and a substituted or unsubstituted C₄-C₂₀ heteroaryl group; and
R¹, R², and R³ are each independently selected from the group consisting of hydrogen and a straight- or branched-chain C₁-C₆ alkyl or alkenyl group.

3. The composition according to claim 1, wherein
R is selected from the group consisting of a C₁-C₁₀ alkyl group or alkoxy group, a C₂-C₁₀ alkenyl or alkenyloxy group, a C₄-C₁₀ dienyl group, a C₃-C₈ cycloalkyl or cycloalkoxy group, a C₆-C₁₂ carbocyclic aryl or aryloxy group, and a C₄-C₁₀ heteroaryl group; and
R¹, R², and R³ are each independently selected from the group consisting of hydrogen and a C₁-C₆ alkyl or alkenyl group.

4. The composition according to claim 1, wherein R¹, R², and R³ are hydrogen.

5. The composition according to claim 1, wherein R is selected from the group consisting of methyl, ethyl, 1,1-dimethylethyl, n-pentyl, phenyl, 3-pyridyl, 4-pyridyl, and ethoxy; and R¹, R², and R³ are hydrogen.

6. The composition according to claim 1, which comprises from 0.01 to 10% by weight of the 2-pyrimidine thioester or thiocarbonate compound.

7. The composition according to claim 1, which comprises from 0.1 to 5% by weight of the 2-pyrimidine thioester or thiocarbonate compound.

8. The composition according to claim 1, which further comprises at least one compound selected from the group consisting of retinol, retinyl esters, tetronic acid, tetronic acid derivatives, hydroquinone, kojic acid, gallic acid, arbutin, 4-hydroxybenzyl alcohol or esters thereof, α-hydroxy acids, niacinamide, pyridoxine, ascorbic acid, vitamin E or derivatives thereof, aloe, salicylic acid, benzoyl peroxide, witch hazel, caffeine, zinc pyrithione, and fatty acid esters of ascorbic acid.

9. A method for brightening skin, comprising topically applying a composition to skin in need of brightening, wherein the composition comprises:
(a) a 2-pyrimidine thioester or thiocarbonate compound having the general formula 1: 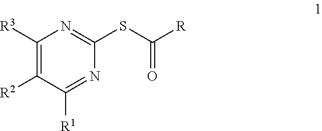
wherein
R is selected from the group consisting of a substituted or unsubstituted, branched- or straight-chain, saturated, unsaturated, or polyunsaturated C₁-C₂₂ alkyl or alkoxy group; a substituted or unsubstituted C₃-C₈ cycloalkyl or cycloalkoxy group; a substituted or unsubstituted C₈-C₂₀ carbocyclic aryl or aryloxy group; and a substituted or unsubstituted C₄-C₂₀ heterocyclic group containing one or more heteroatoms selected from the group consisting of sulfur, nitrogen, and oxygen; and
R¹, R², and R³ are each independently selected from the group consisting of hydrogen; a substituted or unsubstituted, branched- or straight-chain, saturated, unsaturated, or polyunsaturated C₁-C₂₂ alkyl group, wherein the branching and/or substitution of R¹ and R² may connect to form a ring; a substituted or unsubstituted C₃-C₈ cycloalkyl group; a substituted or unsubstituted C₆-C₁₀ carbocyclic aryl group; a substituted or unsubstituted C₄-C₁₀ heterocyclic group containing one or more heteroatoms selected from the group consisting of sulfur, nitrogen, and oxygen; a hydroxyl group; a C₁-C₆ alkoxy group; a carboxyl group; an amino group; a C₁-C₁₅ aminocarbonyl group; a C₁-C₁₅ amido group; a cyano group; a C₂-C₆ alkoxycarbonyl group; a C₂-C₆-alkanoyloxy group; a thiol group; a thioether group; a C₂-C₁₀ dialkylamino group; a C₃-C₁₅ trialkylammonium group; and a halogen, and
(b) a dermatologically acceptable carrier.

10. The method according to claim 9, wherein R is selected from the group consisting of methyl, ethyl, 1,1-dimethylethyl, n-pentyl, phenyl, 3-pyridyl, 4-pyridyl, or ethoxy; and R¹, R², and R³ are hydrogen.

11. The method according to claim 9, wherein the composition comprises from 0.1 to 5% by weight of the 2-pyrimidine thioester or thiocarbonate compound.

12. The method according to claim 1, wherein the composition further comprises at least one compound selected from the group consisting of retinol, retinyl esters, tetronic acid, tetronic acid derivatives, hydroquinone, kojic acid, gallic acid, arbutin, 4-hydroxybenzyl alcohol and esters thereof, α-hydroxy acids, niacinamide, pyridoxine, ascorbic acid, vitamin E and derivatives thereof, aloe, salicylic acid, benzoyl peroxide, witch hazel, caffeine, zinc pyrithione, and fatty acid esters of ascorbic acid.

13. A method for inhibiting melanogenesis, comprising contacting melanocytes with a 2-pyrimidine thioester or thiocarbonate compound having the general formula 1: 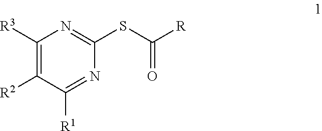
wherein
R is selected from the group consisting of a substituted or unsubstituted, branched- or straight-chain, saturated, unsaturated, or polyunsaturated C₁-C₂₂ alkyl or alkoxy group; a substituted or unsubstituted C₃-C₈ cycloalkyl or cycloalkoxy group; a substituted or unsubstituted C₆-C₂₀ carbocyclic aryl or aryloxy group; or a substituted or unsubstituted C₄-C₂₀ heterocyclic group containing one or more heteroatoms selected from the group consisting of sulfur, nitrogen, and oxygen; and
R¹, R², and R³ are each independently selected from the group consisting of hydrogen; a substituted or unsubstituted, branched- or straight-chain, saturated, unsaturated, or polyunsaturated C₁-C₂₂ alkyl group, wherein the branching and/or substitution of R¹ and R² may connect to form a ring; a substituted or unsubstituted C₃-C₈ cycloalkyl group; a substituted or unsubstituted C₆-C₁₀ carbocyclic aryl group; a substituted or unsubstituted C₄-C₁₀ heterocyclic group containing one or more heteroatoms selected from the group consisting of sulfur, nitrogen, and oxygen; a hydroxyl group; a C₁-C₆ alkoxy group; a carboxyl group; an amino group; a C₁-C₁₅ aminocarbonyl group; a C₁-C₁₅ amido group; a cyano group; a C₂-C₆ alkoxycarbonyl group; a C₂-C₆-alkanoyloxy group; a thiol group; a thioether group; a C₂-C₁₀ dialkylamino group; a C₃-C₁₅ trialkylammonium group; and a halogen.

14. The method according to claim 13, wherein
R is selected from the group consisting of a substituted or unsubstituted, branched- or straight-chain saturated C₁-C₂₂ alkyl group or alkoxy group; a substituted or unsubstituted, branched- or straight-chain C₂-C₂₂ alkenyl or alkenyloxy group; a substituted or unsubstituted, branched- or straight-chain C₄-C₂₂ dienyl group; a substituted or unsubstituted C₃-C₈ cycloalkyl or cycloalkoxy group; a substituted or unsubstituted C₆-C₂₀ carbocyclic aryl or aryloxy group; and a substituted or unsubstituted C₄-C₂₀ heteroaryl group; and
R¹, R², and R³ are each independently selected from the group consisting of hydrogen and a straight- or branched-chain C₁-C₆ alkyl or alkenyl group.

15. The method according to claim 13, wherein
R is selected from the group consisting of a C₁-C₁₀ alkyl group or alkoxy group, a C₂-C₁₀ alkenyl or alkenyloxy group, a C₄-C₁₀ dienyl group, a C₃-C₈ cycloalkyl or cycloalkoxy group, a C₆-C₁₂ carbocyclic aryl or aryloxy group, and a C₄-C₁₀ heteroaryl group; and
R¹, R², and R³ are each independently selected from the group consisting of hydrogen and a C₁-C₈ alkyl or alkenyl group.

16. The method according to claim 13, wherein R¹, R², and R³ are hydrogen.

17. The method according to claim 13, wherein R is selected from the group consisting of methyl, ethyl, 1,1-dimethylethyl, n-pentyl, phenyl, 3-pyridyl, 4-pyridyl, or ethoxy; and R¹, R², and R³ are hydrogen.
